(19) United States
(12) Patent Application Publication
(10) Pub. No.: US 2024/0041644 A1
(43) Pub. Date: Feb. 8, 2024

(54) RIGHT/LEFT EYE DETECTION WITH PROXIMITY SENSOR FOR HORIZONTAL NON-GRAVITATIONAL OPHTHALMIC APPLICATOR

(71) Applicant: TWENTY TWENTY THERAPEUTICS LLC, South San Francisco, CA (US)

(72) Inventor: Timothy Stowe, Alameda, CA (US)

(21) Appl. No.: 18/230,014

(22) Filed: Aug. 3, 2023

Related U.S. Application Data
(60) Provisional application No. 63/394,650, filed on Aug. 3, 2022.
(60) Provisional application No. 63/400,781, filed on Aug. 25, 2022.

(51) Int. Cl. A61F 9/00

(52) U.S. Cl. CPC A61F 9/0008; A61F 2250/0096; A61F 2250/0068

(57) ABSTRACT

An ophthalmic applicator for treating an eye of a user includes a housing and a reservoir, supported by the housing, for containing a liquid for treating the eye. The applicator also includes a nozzle supported by the housing and operatively connected to the reservoir. The nozzle is configured a non-gravitationally directed dose of the liquid. The applicator further includes a proximity sensor configured to identify the eye, left or right, to which the applicator is applying the liquid to the eye of the user.

### RELATED APPLICATIONS

This application claims the benefit of U.S. Provisional Application Ser. No. 63/394,650, filed on Aug. 3, 2022. This application also claims the benefit of U.S. Provisional Application Ser. No. 63/400,781, filed on Aug. 25, 2022. The disclosures of these applications are hereby incorporated herein by reference in their entireties.

### TECHNICAL FIELD

This disclosure relates to an ophthalmic applicator for the self-application of a mist or drops to a user's eye. The applicator includes a proximity sensor for detecting both a working distance from the user's eye and which eye, left or right, is being treated.

### BACKGROUND

Ophthalmic applicators, such as eye droppers or eye misting devices, can be used to apply a variety of fluid treatments, such as lubricants or medications, to a user's eye. Some applicators are specialized in that they are configured to apply fluid treatments in a predetermined shape, pattern, thickness, utilizing specialized nozzles or nozzle arrays to do so. Some of these devices can be electronic, using electromechanical peristaltic means to deliver the fluid at a gentle rate so that the fluid can be applied precisely with velocity and volume. In this manner, the dosage of medication can be closely controlled.

Sometimes users need to treat only one eye or have one eye that needs a different dose of medication or a different medication altogether. For example eye infections usually affect one eye more than another. In cases of glaucoma, one eye may have a more advanced condition. In these cases, it may be useful for the patent or doctor to have a record indicating that the proper amount and/or type of medication was delivered to the correct eye. Additionally, it can be useful to take the decision on which eye to treat with what medication or what dose to apply out of the hands of the user, thus eliminating a source of error.

To facilitate this automated functionality, the applicator needs to “know” whether the application is being delivered to the user's right eye or left eye. With this knowledge, the applicator can apply the spray medication(s) to the proper eye with the proper dosage. Accordingly, as disclosed herein, an ophthalmic applicator is configured to detect the eye—right or left—of the user.

### SUMMARY

As disclosed herein, the ophthalmic applicator can be configured to dose the user's eye via a horizontal spray, drop, or microstream. This configuration eliminates the need to have the user having to tilt their head. As configured, it is natural for users to hold the ophthalmic applicator in the same general position relative to their eye. To detect which eye is being treated, the applicator is outfitted with a proximity sensor, several configurations of which are disclosed herein.

According to one aspect, an ophthalmic applicator for treating an eye of a user includes a housing and a reservoir, supported by the housing, for containing a liquid for treating the eye. The applicator also includes a nozzle supported by the housing and operatively connected to the reservoir. The nozzle is configured a non-gravitationally directed dose of the liquid. The applicator further includes a proximity sensor configured to identify the eye, left or right, to which the applicator is applying the liquid to the eye of the user.

According to another aspect, the proximity sensor can be configured to sense the presence of the user's nose positioned laterally of the housing when the applicator is positioned to treat the eye.

According to another aspect, the proximity sensor can be configured to associate the applicator being used to treat left eye in response to detecting the user's nose. The proximity sensor can also be configured to associate the applicator being used to treat the right eye in response to not detecting the user's nose.

According to another aspect, the proximity sensor can be configured to associate the applicator being used to treat right eye in response to detecting the user's nose, The proximity sensor can also be configured to associate the applicator being used to treat the left eye in response to not detecting the user's nose.

According to another aspect, the proximity sensor can be configured to sense the presence of facial structure laterally of the housing in a direction generally perpendicular to a nozzle axis of the nozzle.

According to another aspect, the proximity sensor can include a miniature surface mounted infrared optical proximity sensor with a built in LED and photodetector.

According to another aspect, the proximity sensor can comprise two different proximity sensors pointing in opposite lateral directions are used for sensing the presence or absence of a user's nose from either side, wherein the applicator can thereby determine which eye is being treated and whether the applicator is held sufficiently close to the user's face to warrant dosage of the liquid to the eye.

According to another aspect, the proximity sensor can include a central photodetector, a left-side LED on one side of the photodetector, and a right-side LED on an opposite side of the photodetector. The LEDS can be configured to produce rays directed away from each other at outward angles with respect to a nozzle axis of the applicator. The proximity sensor can be configured to determine which eye is being treated in response to which of the left-side and right-side LEDs generates the higher magnitude detected signal at the photodetector.

According to another aspect, the applicator can include a controller configured to control the proximity sensor and the application of treatment in response to which eye, left or right, is detected.

According to another aspect, the controller can be configured to compile a treatment log for the treatments applied to the user's eyes and can thus determine if one or both eyes were properly treated.

According to another aspect, the applicator can also include a displacement valve and an actuator configured to actuate the displacement valve. The displacement valve can be configured to, when actuated, force the liquid through the nozzle. The controller can be configured to control actuation of the actuator in response to a user input.

According to another aspect, the reservoir can include a removable reservoir cartridge.

### DRAWINGS

FIG. 1 is a perspective view illustrating an example configuration of an ophthalmic applicator.

FIG. 2 is a right side view of the ophthalmic applicator of FIG. 1.

FIG. 3 is a left side view of the ophthalmic applicator of FIG. 1.

FIG. 4 is a front view of the ophthalmic applicator of FIG. 1.

FIG. 5 is a perspective view illustrating the ophthalmic applicator being held by a user.

FIGS. 6A and 6B are schematic side and front views, respectively, illustrating the main functional components of the ophthalmic applicator.

FIGS. 7A and 7B are front and side views, respectively, illustrating an ophthalmic applicator positioned relative to a user, according to another example configuration.

FIG. 8 is a front view illustrating an ophthalmic applicator positioned relative to a user, according to yet example configuration.

FIGS. 9A and 9B are front and side views, respectively, illustrating certain components of the ophthalmic applicator, according to a further example configuration.

FIG. 9C is a magnified schematic partial view of the ophthalmic applicator of FIGS. 9A and 9B, illustrating certain features.

FIG. 9D is a schematic top view illustrating the use of the ophthalmic applicator of FIGS. 9A-9C.

### DESCRIPTION

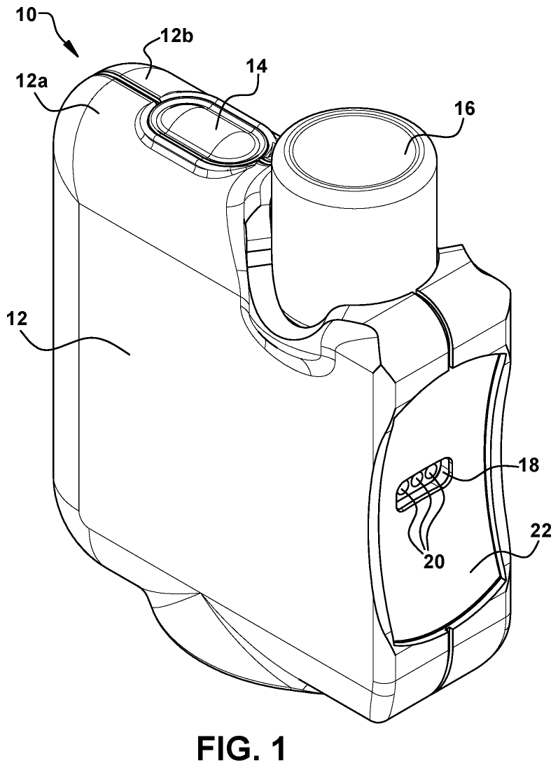

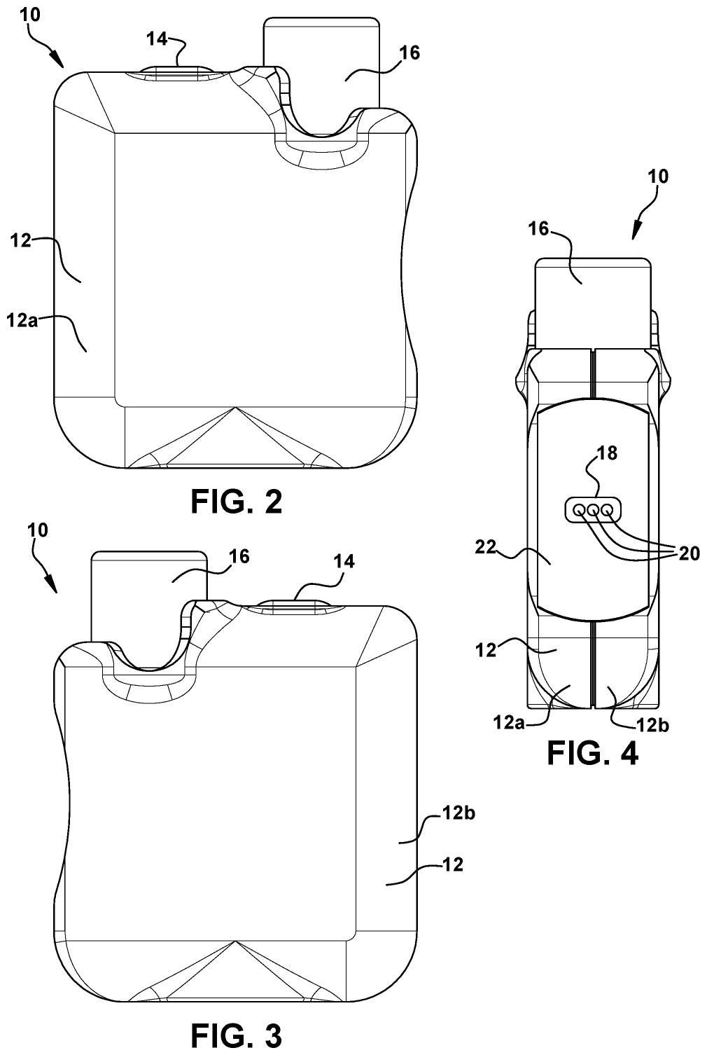

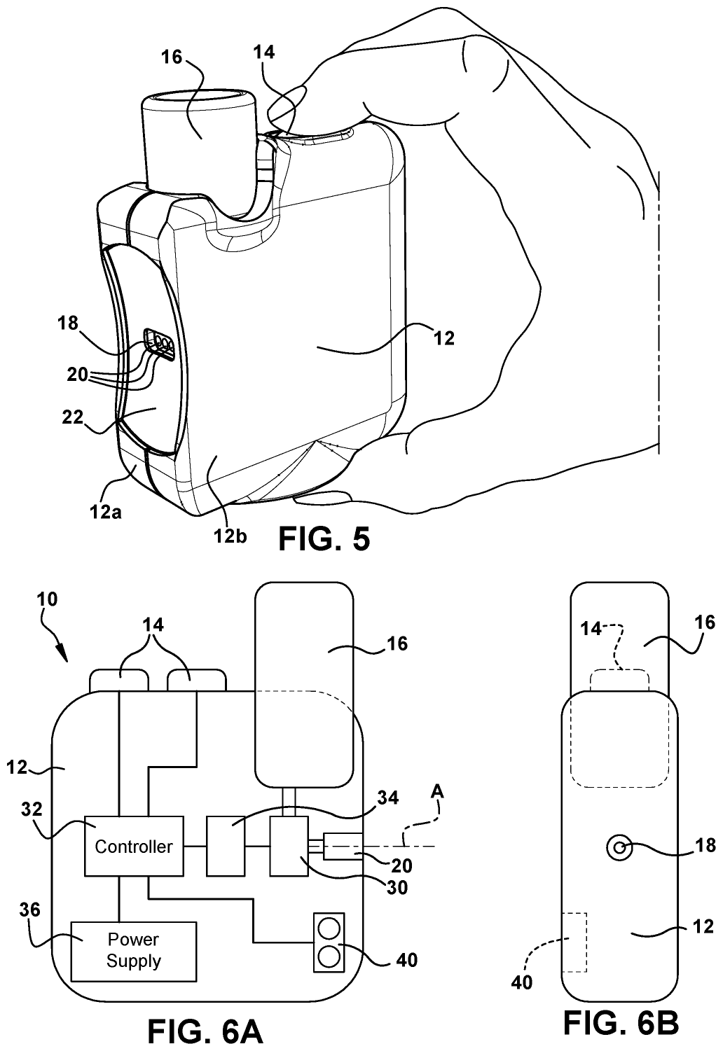

An ophthalmic applicator is configured to allow users to apply a highly controlled ophthalmologic mist to their eyes. An example configuration of an ophthalmic applicator 10 is illustrated in FIGS. 1-5. The ophthalmic applicator 10 includes a housing 12 that includes interconnected first and second housing halves 12a, 12b. The ophthalmic applicator 10 is configured for handheld use by a user, as shown in FIG. 5, and includes one or more control buttons 14 for controlling the operation of the applicator.

The ophthalmic applicator 10 includes a removable reservoir cartridge 16 in which an ophthalmologic fluid, for example a spray medication solution, is stored. The ophthalmologic fluid is dispensed from one or more nozzles 20 (see FIGS. 1, 4, and 5) through an opening 18 in the housing. The opening 18 is formed in a concave surface 20 of the housing 12.

Not shown in FIGS. 1-4 is a sensor for determining the position of the ophthalmic applicator 10 and the side of the user's face, left or right, at which the applicator is positioned. Through this, the ophthalmic applicator 10 can determine which eye, left or right, is being treated. Advantageously, this can allow for different treatments to be applied to different eyes. For example, the spray pattern and/or the volume of ophthalmologic fluid can be custom tailored to each specific eye.

### Single Sensor Configuration

A schematic illustration of an example configuration the ophthalmic applicator 10 is illustrated in FIGS. 6A and 6B. As shown, the applicator housing 12 supports a single nozzle 18 through which the ophthalmologic fluid is directed for application to the user's eye. The applicator 10 includes a displacement valve 30 for pushing the liquid from the reservoir cartridge 16 through the nozzle 18. The reservoir cartridge 16 is swappable so that the applicator 10 can be used to apply different treatments. The housing 12 also supports a proximity sensor 40 that is utilized to identify the eye, left or right, that the applicator 10 is treating.

In the example configuration of FIGS. 6A and 6B, the ophthalmic applicator 10 includes two control buttons 14 that allow the user to operate the unit. The control buttons 14 provide inputs to a controller 32. The controller 32 is configured to control the operation of an actuator 34 configured to actuate the displacement valve 30 to move the fluid through the nozzle 18. The controller 32 is also configured to control the operation of the proximity sensor 40 to determine the identity of the eye being treated. Additionally, the controller 40 includes memory components for storing programmed instructions, settings, displacement valve controls, application history, medication information, dosage settings, history logs, etc. A power supply 36, such as a battery or battery pack, powers the controller 32, the actuator 34, and the proximity sensor 40.

The controller 32 utilizes the proximity sensor 40 to detect the presence of the user's facial features, such as the nose or cheek, during use. In the configuration of the ophthalmic applicator 10 illustrated in FIGS. 6A and 6B, the proximity sensor 40 is mounted on one lateral side of the applicator and is directed laterally of the unit, transverse to the nozzle axis A.

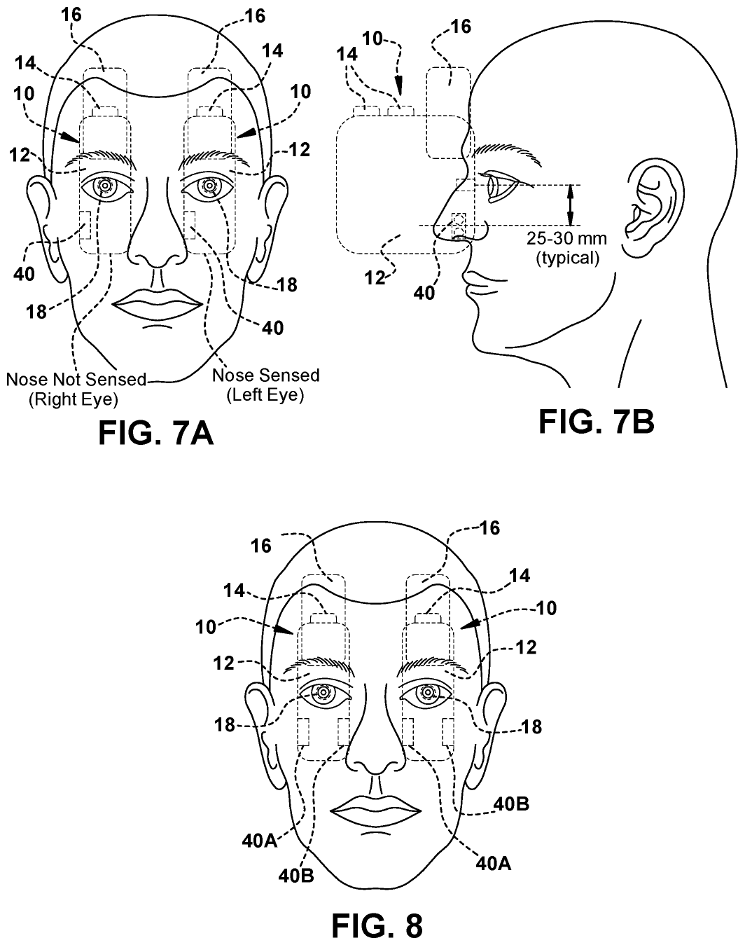

Held with the user's fingers on the control buttons 14 (see FIG. 5), the ophthalmic applicator 10 is held in place in front of the eye being treated. Held in place, the proximity sensor 40 is configured to detect the presence of the user's nose laterally of the applicator. Some proximity sensors, such as infrared proximity sensors, are even insensitive to whether or not a face covering or mask is worn over the face and nose. This is shown in FIGS. 7A and 7B.

Because the proximity sensor 40 is mounted on one side of the applicator 10 only, the side of the face and, thus, the left/right eye identification, can be determined simply as corresponding to whether or not the nose is sensed from a comparison of a reflected signal to a threshold value. In the example configuration of FIGS. 7A and 7B, the side of the applicator housing 12 on which the proximity sensor 40 is mounted associates the detected presence of the user's nose with being positioned in front of the left eye, and the lack of the nose being detected with being positioned in front of the right eye. Because the tip of the human nose is typically 25-30 mm below the center of the eye, the proximity sensor 40 can be precisely configured and arranged to detect the nose. During applicator 10 use, the nozzle 18 will be positioned generally centered on the eye, so the proximity sensor 40 can be positioned on the applicator so that it covers the area where the nose would be positioned. Whether or not the nose is detected is therefore determinative of the side of the face—left or right—where the eye is located.

As shown in FIGS. 7A and 7B, when the ophthalmic applicator 10 is positioned over the user's left eye, the proximity sensor 40 faces and detects the user's nose. The applicator 10 therefore determines that it is being used to treat the user's left eye. When the applicator 10 is positioned over the user's right eye, the proximity sensor 40 faces away from the nose, so it is not detected. The applicator 10 therefore determines that it is being used to treat the user's right eye.

In one example, the proximity sensor 40 can be an infrared optical proximity sensor directed laterally. In this instance, the proximity sensor can be an infrared optical sensor that measures a reflected optical signal from a person's nose. These devices are well-known and cheap. As such, they offer an effective alternative to complicated and comparatively expensive cameras with machine learning to detect edge features indicative of the eye.

The proximity sensor 40 is ideally formed and packaged monolithically in a surface mount package with an invisible infrared light emitted diode (LED). In one particular configuration, the LED can emit light at a wavelength of 940 nm, which has very little back reflection sensitivity to skin color or mask colors, and can utilize LED focusing optics so that the sensing area is limited and targeted. The LED can be mounted next to a photodetector sensor that captures reflected and scattered light from nearby surface(s). The photodetector typically filters out all background light outside the wavelength emitted by the LED, as well as all noise outside the pulsed carrier frequency of the LED.

The proximity sensor 40 can have alternative configurations, such as ultrasonic or capacitive sensor configurations, although these are typically more costly and less discriminating of the targeted area.

The proximity sensor 40 and the detection of the left/right eye allows for several functions. For example, this allows for different doses and/or different medications to be applied to the eyes. Additionally, because the applicator 10 determines which eye is being treated, it can generate an application log that identifies the eye being treated, the dose, the type of medication, and the date/time of the application. The applicator 10 can also be programmed with an application schedule and remind the user when applications are required. The applicator 10 can also instruct the user during use, for example alerting them if/when the user is attempting to treat the wrong eye.

### Two Sensor Configuration

In addition to the configuration of FIGS. 7A and 7B, where only one laterally directed proximity sensor 40 is used to detect the nose, the ophthalmic applicator 10 can include a pair of proximity sensors to be used pointed in opposite directions, so that the left or right eye application can be determined. This is shown in FIG. 8. Essentially, the ophthalmic applicator 10 of FIG. 8 identifies the eye being treated by determining which proximity sensor, 40A or 40B, measures a value above a threshold indicating the presence of the user's nose.

The two-sensor configuration of FIG. 8 can offer several benefits. For example, if neither proximity sensor senses the nose, then the ophthalmic applicator 10 can be assumed to be too far from the patient's face. This can be used to inhibit delivery of a spray dosage until such time that the proper positioning, i.e., nose detection, is achieved. The lack of sensing the user's nose can also be used to disable some features, such as blink detection, until such a time frame at which the presence of a person's nose is detected. This can be advantageous in making sure the initial motion of the applicator towards a person's eye is stable first, and then blink detection algorithms can be turned on.

### Forward Facing Angled Sensor Configuration

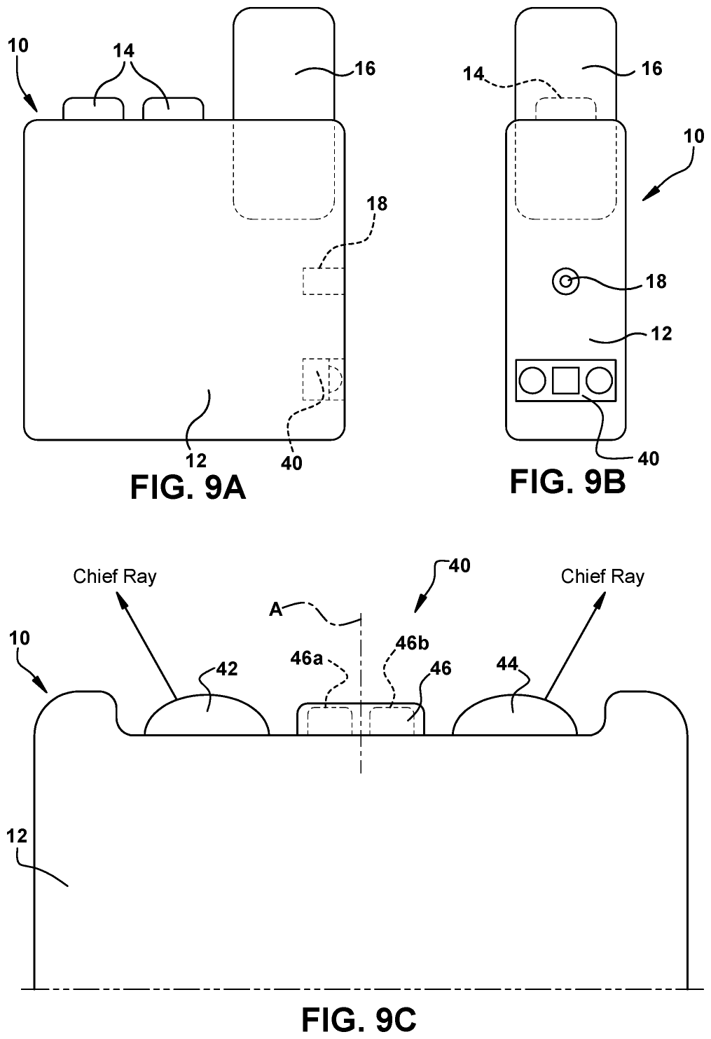

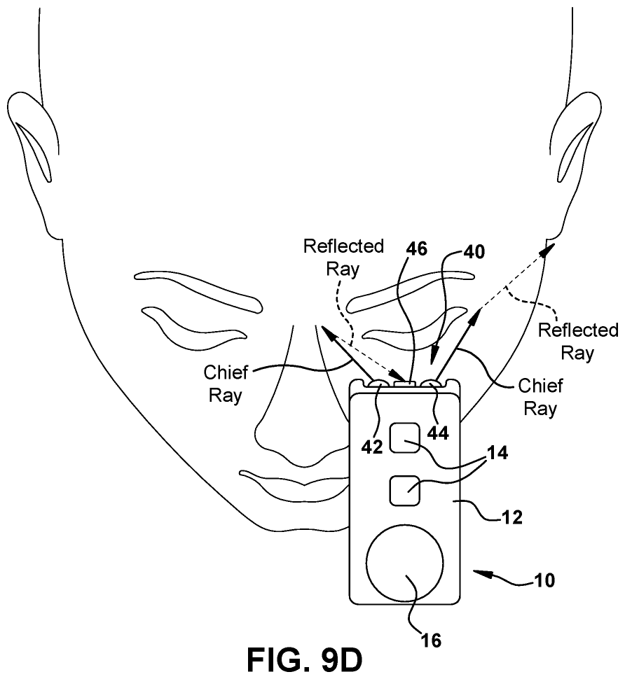

Another example configuration is illustrated in FIGS. 9A-9D. In this further embodiment, it is possible to configure the proximity sensor 40 to include a pair of LEDs 42, 44 arranged in a forward-facing, outward angled configuration. The sensor assembly 40 also includes one or more photodetectors 46 configured to detect reflected light signals from the LEDs 42, 44. In one example configuration, the proximity sensor 40 can include an individual photodetector, indicated in dashed lines at 46a and 46b in FIG. 9C, associated with each LED 42, 44, respectively. In an other example configuration, the sensor assembly can include a central photodetector, indicated in solid lines at 46 in FIGS. 9B-9D, for detecting reflected light signals from both LEDs 42, 44.

As shown in FIG. 9C, the left and right interrogator LEDs 42, 44 are configured to direct their chief light rays at an angle that is outward with respect to the nozzle axis A. In this configuration, chief rays are angled toward the user's face when held in the application position with the nozzle directed toward the eye, as shown in FIG. 9D. In this configuration, the LEDs 42, 44 can be operated sequentially, in an alternating fashion, with the central collecting photodetector 46 being used to detect the reflected beam from each LED. As shown in FIG. 9D, the amount of light reflected back to the photodetector 46 by each LED 42, 44 will depend on which side, left or right, the applicator is positioned. In either position, the chief ray of the LED 42, 44 directed temporally, i.e., toward the user's cheek and ears will be scattered and will result in little reflection back to the photodetector 46. Conversely, the chief ray of the LED 42, 44 directed nasally, i.e., toward the user's eye and nose will be highly reflected back to the photodetector 46. Of course the degree of scattering and reflection for each LED will vary depending on the anatomy and facial structure of the user. Nevertheless, for a properly positioned ophthalmic applicator 10, the LED beam directed toward the user's nose will produce a greater degree of reflection, and therefore can be considered indicative of the eye, left or right, being treated.

In this configuration, the LEDs 42, 44 can be turned on and off independently and sequentially to interrogate a person's face such that the photodetector 46 can determine the side of the user's face being treated by the applicator. In volume, this configuration can reduce the cost of implementing two independent proximity sensors (see sensors 46a and 46b). At the same time, the sensor assembly 40 can provide feedback on whether the user's face is in range so that other functionality, such as blink detection, can be activated, or so that the spray, drop, or micro-stream application can be activated.

1. An ophthalmic applicator for treating an eye of a user, comprising:
a housing;
a reservoir, supported by the housing, for containing a liquid for treating the eye;
a nozzle supported by the housing and operatively connected to the reservoir and being configured to direct a non-gravitationally directed dose of the liquid; and
a proximity sensor configured to identify the eye, left or right, to which the applicator is applying the liquid to the eye of the user.

2. The applicator recited in claim 1, wherein the proximity sensor is configured to sense the presence of the user's nose positioned laterally of the housing when the applicator is positioned to treat the eye.

3. The applicator recited in claim 1, wherein the proximity sensor is configured to associate the applicator being used to treat left eye in response to detecting the user's nose, and to associate the applicator being used to treat the right eye in response to not detecting the user's nose.

4. The applicator recited in claim 1, wherein the proximity sensor is configured to associate the applicator being used to treat right eye in response to detecting the user's nose, and to associate the applicator being used to treat the left eye in response to not detecting the user's nose.

5. The applicator recited in claim 1, wherein the proximity sensor is configured to sense the presence of facial structure laterally of the housing in a direction generally perpendicular to a nozzle axis of the nozzle.

6. The applicator recited in claim 1 where the proximity sensor comprises a miniature surface mounted infrared optical proximity sensor with a built in LED and photodetector.

7. The applicator recited in claim 1, wherein two different proximity sensors pointing in opposite lateral directions are used for sensing the presence or absence of a user's nose from either side, wherein the applicator can thereby determine which eye is being treated and whether the applicator is held sufficiently close to the user's face to warrant dosage of the liquid to the eye.

8. The applicator recited in claim 1, wherein the proximity sensor comprises a central photodetector, a left-side LED on one side of the photodetector, and a right-side LED on an opposite side of the photodetector, wherein the LEDS are configured to produce rays directed away from each other at outward angles with respect to a nozzle axis of the applicator, wherein proximity sensor is configured to determine which eye is being treated in response to which of the left-side and right-side LEDs generates the higher magnitude detected signal at the photodetector.

9. The applicator recited in claim 1, further comprising a controller configured to control the proximity sensor and the application of treatment in response to which eye, left or right, is detected.

10. The applicator recited in claim 9, wherein the controller is configured to compile a treatment log for the treatments applied to the user's eyes and can thus determine if one or both eyes were properly treated.

11. The applicator recited in claim 9, further comprising a displacement valve and an actuator configured to actuate the displacement valve, wherein the displacement valve is configured to, when actuated, force the liquid through the nozzle, and wherein the controller is configured to control actuation of the actuator in response to a user input.

12. The applicator recited in claim 1, wherein the reservoir comprises a removable reservoir cartridge.
